(19) United States
(12) Patent Application Publication
(10) Pub. No.: US 2013/0053764 A1
(43) Pub. Date: Feb. 28, 2013

(54) ELECTRICAL PUMP FOR BREAST MILK

(71) Applicant: Reinhold Jaeger-Waldau, Scheessel (DE)

(72) Inventor: Reinhold Jaeger-Waldau, Scheessel (DE)

(73) Assignee: MAPA GMBH, Zeven (DE)

(21) Appl. No.: 13/578,156

(22) PCT Filed: May 2, 2011
(86) PCT No.: PCT/EP2011/002168

§ 371 Date: Aug. 16, 2012

(30) Foreign Application Priority Data
May 3, 2010 (DE) 10 2010 019 041.

(51) Int. Cl. A61M 1/06

(52) U.S. Cl. 604/74

(57) ABSTRACT

An electrical pump for breast milk, having an electric motor, a pump, at least one suction cup, a suction line connecting the suction cup to the pump, an outlet for breast milk connected to the suction cup, a venting line connected to the suction cup, an electrically activated venting valve in the venting line, an electronic control system which adjusts the speed of the electric motor and the electrically activated venting valve to switch the electric motor on and off to allow the electric motor to run at a desired speed and to operate the electric motor continuously and/or in cycles, in which the electric motor is alternately switched on and off, and the electronic control system opens the venting valve when the electric motor is switched off, and closes the venting valve when the electric motor is switched on.

### CROSS-REFERENCE TO RELATED APPLICATIONS

This application is a U.S. National Stage Application which claims priority to PCT/EP2011/002168, filed May 2, 2011, which claims priority to DE 10 2010 019 041.1 filed May 3, 2010.

### STATEMENT REGARDING FEDERALLY SPONSORED RESEARCH

Not applicable

### BACKGROUND OF THE INVENTION

The present invention relates to an electric breast milk pump.

Breast milk pumps serve for pumping away breast milk. For this purpose, they have at least one suction cup, which is set onto the mother's breast. A negative pressure is applied to the suction cup in order to withdraw the milk from the mother's breast. This negative pressure will also be designated as “suction underpressure” in the following. The suction cup is connected to a reservoir, which receives the breast milk that is suck off. The negative pressure is generated by means of a pump, which is driven manually or by means of an electric motor. Electric breast milk pumps driven by means of an electric motor are known, which have an electronic control system which controls certain pumping sequences. In these pumping sequences, the pump is switched on and off during defined periods in order to stimulate the flow of milk. The magnitude of the negative pressure (called underpressure in the following) can be adjusted.

From the document EP 1 587 340 A2, an electric breast milk pump is known which is coupled to a gyro valve. The gyro valve comprises a centrifugal control system, which opens a spring-tensioned venting valve when the electric motor is switched off. The venting valve vents the suction cup in order to break down the underpressure in the rest periods of the pumping. When the electric motor is running, the venting valve is closed by spring force, so that the underpressure in the suction cup is built up in the pumping phases. During the pumping sequence, the pump is switched on and off several times. The underpressure in the pumping phases can be adjusted with the aid of a setting dial, which is connected to an adjustment valve via which the suction cup is vented permanently. Depending on the setting of this valve, a more or less great underpressure is built up in the suction cup.

In the known electric breast milk pump, the electric motor runs always with maximum speed. As a consequence, the breast milk pump has a high level of noise and high energy consumption. Further, this has the effect to shorten the lifetime of the electric motor. The centrifugal control system is also a reason for the high energy consumption, a part of the drive power being allotted to it. Further, the high energy consumption is due to the fact that the underpressure is broken down by the gyro valve immediately after switching off the motor, so that the spin-out phase of the motor is not used for the development of the underpressure. In turn, the lifetime of the electric motor is shortened by its longer running times. The course of the underpressure at the beginning and at the end of the pumping phase is fixedly set by the construction of the gyro valve. Special underpressure courses which extra stimulate the milk flow cannot be programmed.

### BRIEF SUMMARY OF THE INVENTION

Starting from this, the present invention is based on the task to provide an electric breast milk pump which has a lower level of noise and wear, as well as less energy consumption.

This task is achieved by an electric breast milk pump with the features of claim 1.

The electric breast milk pump according to the present invention has

an electric motor,

a pump driven by the electric motor,

at least one suction cup,

a suction line connecting the suction cup to the pump,

an outlet for breast milk, communicatingly connected to the suction cup,

a venting line, communicatingly connected to the suction cup,

an electrically activated venting valve in the venting line,

means for switching on and off the electric motor,

means for adjusting the speed of the electric motor,

an electronic control system, which is connected to the means for switching on and off, the means for adjusting the speed, the electric motor and the electrically activated venting valve and which is configured to switch the electric motor on and off depending on an activation of the means for switching on and off, to allow the electric motor to run at a speed according to the setting of the means for adjusting the speed, and to operate the electric motor continuously and/or to operate the electric motor in cycles, in which the electric motor is alternately switched on and off, wherein the electronic control system opens the venting valve when the electric motor is switched off, and closes it when the electric motor is switched on.

In the breast milk pump of the present invention, the suction underpressure is adjusted by the electronic control system by means of the motor speed. This has the advantage that the electric motor consumes less current on average, and that it runs more quietly than if it were always operated at maximum speed. The pumping power for the venting flow of the conventional breast milk pump has not to be provided by the electric motor. The wear of the electric motor is reduced, because on average, it works with reduced speed. Moreover, the electric motor does not have to provide the power for the operation of a gyro valve. In fact, this saving is more or less compensated by the energy consumption of the electrically activated venting valve. But the electrically activated venting valve has the advantage that it can be switched off by the electronic control system with an arbitrary delay with respect to the electric motor, so that the spin-out phase of the pump can be used for the build-up of the suction underpressure. Therefore, shorter switched-on phases of the electric motor are sufficient for the breast milk pump to generate a certain suction underpressure. Even this reduces the energy consumption, the running noise and the wear. On the other hand, the venting valve can be opened concomitantly with the electric motor or before switching off the electric motor, for instance in order to influence the course of the suction underpressure at the end of the pumping phase. Further, the venting valve can be closed at an arbitrary moment with respect to the switch-on moment of the electric motor, in particular in order to shape the course of the suction underpressure at the beginning of the pumping phase. In order to influence the course of the suction underpressure at the beginning and/or the end of the pumping phase, the change of the speed of the electric motor can also be controlled by the electronic control system. The milk flow can be stimulated in an advantageous manner by the design of the course of the suction underpressure.

The advantageous effects mentioned above are effective when the breast milk pump is operated continuously, so that it runs permanently between the moments of switching on and off. The advantageous effects of the present invention are particularly effective when the electric motor is operated in cycles, in which it is switched on and off repeatedly. The present invention is related to breast milk pumps that are operated only continuously or only in cycles, but also to breast milk pumps that can be operated continuously as well as in cycles.

Of course, the breast milk pump has a power supply, which feeds the electric motor, the venting valve and the electronic control system. In this, it can be dealt with a battery, an accumulator, a charger or respectively a mains adapter in particular. Combinations of the mentioned power supplies are possible.

According to one embodiment, the electronic control system opens the venting valve with a time delay after switching off the electric motor. The time delay may be for instance 0.5 seconds, in order to utilize the spin-out phase of the electric motor for the build-up of the suction underpressure.

The electric motor is preferably a DC motor with brushes and commutators. Such motors, which are designed for a working life of some 100 hours of function, are available in compact construction and with suitable power dimensions. By the present invention, the lifetime of the electric motors is prolonged, because they are not energized so much, do not run with such a high speed and not for so long as is the case in the state of the art. Through this, the wear of the brushes of a DC motor with brushes is reduced in particular.

According to a preferred embodiment, the venting valve is an electromagnetic valve. Further preferred, the electromagnetic valve is a solenoid valve. But the use of other electrically activated valves is also possible, for instance of a valve activated by a piezo drive.

According to a further embodiment, the electronic control system comprises a circuit board with a microcomputer, in which different programs are stored which can be selected by optional placement of jumpers at different positions of the circuit board. The jumpers connect strip conductors of the printed circuit board, which are connected to the micro computer. Depending on which strip conductors are connected, the micro computer is operated in different switching conditions wherein it performs different programs. By setting the jumpers, it is possible to select desired programs of the pump in its production. Through this, a family of breast milk pumps with different pumping sequences can be provided. Just only the variable attachment of jumpers is necessary for this. For instance, the jumpers may be wire bridges that are soldered into the circuit board.

Thus, it is for instance possible to set the pumping sequences of the breast milk pump by a certain placement of jumpers such that, in a first step, it performs 60 pumping cycles per minute, wherein each pumping cycle comprises a pumping phase of 0.5 s and a venting phase of 0.5 s, and in a second step, 30 pumping cycles per minute, wherein each pumping cycle comprises a pumping phase of 1 s and a venting phase of 1 s. By repeated activation of the means for switching on and off, the pumping sequence can be begun with the first step, and the transition from the first step to the second step and switching off the pump can be controlled. Further, by just simply placing jumpers, it is possible to set the microcomputer such that the breast milk pump performs 30 pumping cycles per minute permanently, wherein each pumping cycle comprises a pumping phase of 1 s duration and a venting phase of 1 s duration. The pumping sequences can be started and stopped by activating the means for switching on and off.

According to a further embodiment, the different pumping cycles differ in one or several of the following parameters: duration of the pumping phases, duration of the venting phases, magnitude of the suction underpressure, magnitude of a remaining underpressure in the venting phases, course of the rise of the suction underpressure in the pumping phases, course of the decay of the suction underpressure in the venting phases. It is possible to influence the course of the suction underpressure by controlling the pump's rotational speed and the moment of closing and opening the electrically activated venting valve.

According to a preferred embodiment, the pump is a membrane pump. By means of a membrane pump, the necessary suction underpressures can be built up in a short time with compact construction. The suction underpressures are preferably in the range of 330 to 100 mbar. In the pumping phases, these suction underpressures can be built up by means of a membrane pump within a time in the range of seconds, or even below.

The outlet of the breast milk pump is preferably arranged in an opening of a bottle or another reservoir for breast milk. According to a further embodiment, a milk outlet valve exists between the suction cup and the outlet for breast milk. The milk outlet valve opens when milk accumulates above it, in order to discharge the milk into a bottle for instance.

According to a further embodiment, the milk outlet valve is a duckbill valve. The duckbill valve is a valve having a V-shaped cross section, with a slit at the contact line between the legs of the V. The milk outlet valve opens when the duckbill valve is sufficiently filled with breast milk between the legs of the V, in order to discharge the same into a bottle for instance.

According to one embodiment, a floater valve is arranged between the suction cup and the pump. The floater valve closes through floating up against a valve seat of the breast milk pump only then when the milk is not discharged sufficiently fast enough into the reservoir/the bottle via the milk outlet valve. This is the case when the space between milk outlet valve and suction cup is filled. Upon “complete sealing”, the floater valve prevents liquid from being aspirated into the pump. Dried milk can agglutinate and block the inlet- and outlet valves of the pump.

According to a further embodiment, the breast milk pump comprises a connection channel, on which the milk outlet valve is arranged at the downside and the floater valve at the upside, wherein the suction cup is connected between milk outlet valve and floater valve, and the suction line is attached to the floater valve at the upside. The connection channel is preferably connected to a lid for putting onto a bottle. This lid is preferably a screw lid.

Further preferred, the lid has a venting channel for venting a bottle on whose bottle opening the lid is fixed.

According to a further embodiment, the electric motor, the pump, the electronic control system, the venting valve and a power supply are integrated into a casing.

According to a further embodiment, the casing is rigidly connected to the connection channel, or the connection channel is at least partially integrated into the casing, respectively.

### BRIEF DESCRIPTION OF THE SEVERAL VIEWS OF THE DRAWINGS

FIG. 1 is a partial cross-sectional side view, partial schematic vertical section view of one embodiment of the present invention.

FIG. 2 is a detail vertical section view of one embodiment of a venting valve of the present invention.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated.

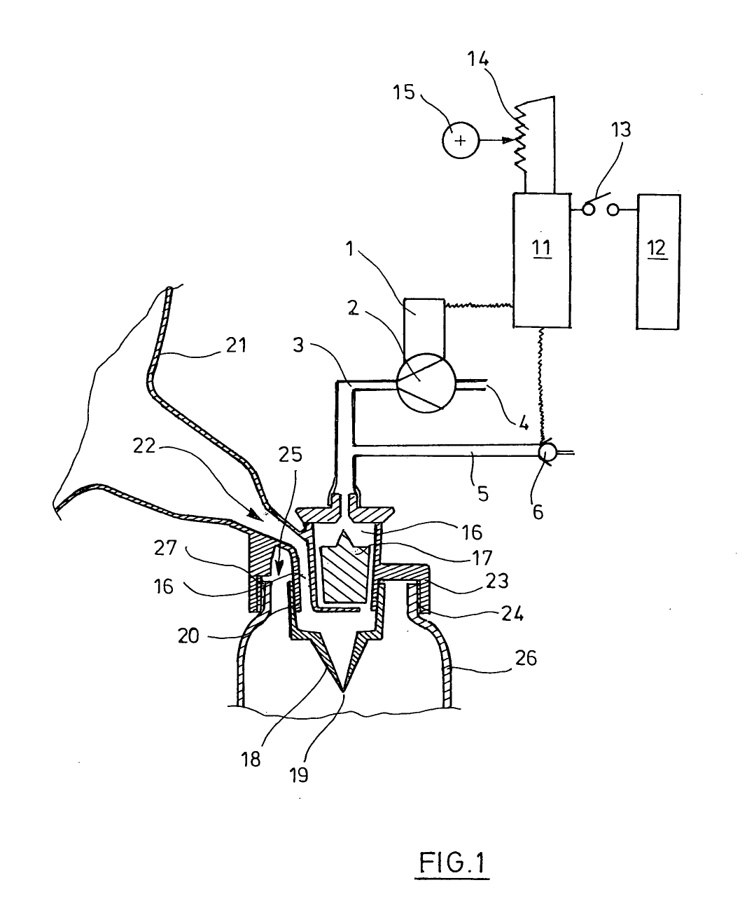

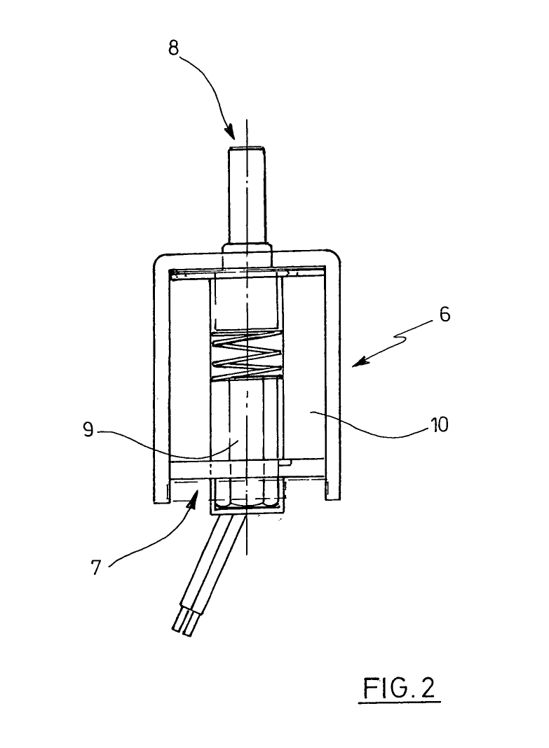

The present invention will be explained by way of the attached drawings, which show in FIG. 1 a breast milk pump of the present invention in a rough schematic vertical section, and in FIG. 2 a venting valve in a vertical section.

According to FIG. 1, the breast milk pump has an electric motor 1 designed as a DC motor with brushes and commutator, and a pump 2 mechanically coupled thereto, which is configured as a membrane pump. A suction line 3 is connected to the inlet of pump 2. The outlet of pump 2 is connected to an outlet 4 for air.

The suction line 3 is connected to a venting line 5, in which is arranged an electrically activated venting valve 6, which vents towards the atmosphere. According to FIG. 2, the venting valve 6 is designed as a solenoid valve with inlet 7, outlet 8, movable ferromagnetic valve body 9 and electromagnet 10.

According to FIG. 1, the breast milk pump comprises an electronic control system 11 and a power supply 12. The electronic control system 11 is connected to an on-off switch 13 and means for adjusting the speed of the electric motor 1, which have a setting dial 15.

The suction line 3 is connected to the upper end of a connection channel 16. A floater valve 17 is arranged in the upper end of the connection channel 16.

Further, the connection channel 16 is equipped at its lower end with a milk outlet valve 18 in the form of a duckbill valve with a valve slit 19 at its lower end. This valve is thrust onto a neck-shaped pipe part 20 on the lower end of the connection channel 16, or inserted into the same.

Further, the connection channel 16 is laterally connected rigidly to a suction cup 21. The suction cup 21 enlarges towards the outside and is connected to the connection channel 16 at its inner end via a passage opening 22.

At the outside, the connection channel 16 is connected to a screw ring 23, which can be screwed to a screw thread 24 on the bottle opening 25 of a bottle 26, so that the milk outlet valve 18 projects into the bottle opening 25. The screw ring 23 has an integrated air channel 27.

By activating the on/off switch 13, the breast milk pump can be switched on. The electronic control system 11 controls the speed of the pump 2 depending on the pump speed set by way of the means for adjusting 14. The electronic control system 11 controls the electric motor 1 such that the pump 2 is switched on and off again during a certain number of pumping cycles in a time span. In the pumping phases, the electronic control system 11 switches on the electric motor 1 and closes the electrically activated venting valve 6. When the pump 2 is switched off in the venting phases, the electronic control system 11 opens the electrically activated venting valve 6.

In the pumping phases, an underpressure in built up in the connection channel 16 via the suction line 3 and the floater valve 17. As a consequence, milk is sucked off from a mother's breast by the suction cup 21. This milk accumulates on the milk outlet valve 18 and drains down into the bottle 26 when a sufficient amount has been aspirated to open the valve slit 19 at the lower end of the milk outlet valve 18.

After a given number of pumping cycles or after switching off the means for switching on and off 13, the pump 2 is switched off.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. Electrical pump for breast milk, comprising:
an electric motor;
a pump driven by the electric motor;
at least one suction cup;
a suction line connecting the suction cup to the pump;
an outlet for breast milk, communicatingly connected to the suction cup;
a venting line, communicatingly connected to the suction cup;
an electrically activated venting valve in the venting line;
means for switching on and off the electric motor;
means for adjusting the speed of the electric motor; and
an electronic control system, which is connected to the means for switching on and off, the means for adjusting the speed, the electric motor and the electrically activated venting valve and which is configured to switch the electric motor on and off depending on an activation of the means for switching on and off, to allow the electric motor to run at a speed according to the setting of the means for adjusting the speed, and to operate the electric motor continuously and/or to operate the electric motor in cycles, in which the electric motor is alternately switched on and off, wherein the electronic control system opens the venting valve when the electric motor is switched off, and closes it when the electric motor is switched on.

2. The breast milk pump according to claim 1, wherein the electronic control system opens the venting valve with a time delay after switching off the electric motor or before switching off the electric motor.

3. A breast milk pump according to claim 1, wherein the venting valve is an electromagnetic valve.

4. The breast milk pump according to claim 3, wherein the electromagnetic valve is a solenoid valve.

5. A breast milk pump according to any one of claims claim 1, wherein the electronic control system comprises a circuit board with a microcomputer, in which different programs are stored which can be selected by optional placement of jumpers at different positions of the circuit board.

6. The breast milk pump according to claim 5, wherein the different programs of an electronic control system control different pumping sequences.

7. A breast milk pump according to claim 5, wherein the different pumping cycles differ in at least one of duration of the pumping phases, duration of the venting phases, magnitude of the suction underpressure, magnitude of a remaining underpressure in venting phases, course of the rise of the suction underpressure in the pumping phases, or course of the decay of the suction underpressure in the venting phases.

8. A breast milk pump according to claim 1, wherein the pump is a membrane pump.

9. A breast milk pump according to claim 1, wherein a milk outlet valve exists between the suction cup and the outlet for breast milk, which opens when milk accumulates above it.

10. The breast milk pump according to claim 9, wherein the milk outlet valve is a duckbill valve.

11. A breast milk pump according to claim 9, wherein a floater valve is arranged between suction cup and pump, which closes when milk above the milk outlet valve rises up to the floater valve.

12. A breast milk pump according to any one of claims claim 1, having a connection channel, on which the milk outlet valve is arranged at the downside and the floater valve at the upside, wherein the suction cup is connected between milk outlet valve and floater valve, and the suction line is attached to the floater valve at the upside.

13. The breast milk pump according to claim 12, wherein the connection channel is connected at its outside to a lid for putting onto a bottle.

14. The breast milk pump according to claim 13, wherein the lid has a venting channel for venting a bottle.

15. A breast milk pump according to claim 1, wherein the electric motor, the pump, the electronic control system, the venting valve, an electronic power supply and the connection channel are at least partially integrated into a casing.
